(19) United States
(12) Patent Application Publication
(10) Pub. No.: US 2008/0065129 A1
(43) Pub. Date: Mar. 13, 2008

(54) Tissue morcellating device

(75) Inventors: Kester J. Batchelor, Minneapolis, MN (US); Alistair I. Fleming, Usk (GB); Rhodri G. James, Heath (GB); Thomas R. K. Edwards, Llanishen (GB)

Correspondence Address: NIXON & VANDERHYE, PC, 901 NORTH GLEBE ROAD, 11TH FLOOR, ARLINGTON, VA, 22203, US

(73) Assignee: Gyrus Medical Limited, Cardiff (GB)

(21) Appl. No.: 11/523,754

(22) Filed: Sep. 20, 2006

(30) Foreign Application Priority Data
Sep. 7, 2006 (GB) 0617600.2

(51) Int. Cl. A61B 17/32

(52) U.S. Cl. 606/172

(57) ABSTRACT

A tissue morcellating device (1) is provided for morcellating tissue within a body cavity of a patient. The morcellating device (1) comprisies a hollow tube (3) having a distal end portion, and tissue cutting means (5) located at the distal end of the tube, such that, when relative movement is initiated between the tube and the tissue, a core of severed tissue is formed within the tube for removal from the body cavity of the patient through the hollow tube. The hollow tube (3) is provided with a stop mechanism (7) which is longitudinally adjustable with respect to the hollow tube (3), the stop mechanism being provided with a shoulder (8) for limiting the longitudinal insertion of the tube into the patient's body.

### BACKGROUND OF THE INVENTION

This invention relates to a tissue morcellating device for use in the bulk removal of tissue, as in a laparoscopic hysterectomy.

In a laparoscopic hysterectomy, the body of the uterus is resected from the stump or fundus, and then removed from the operative site. To enable the uterus to be removed through a limited surgical opening, it is desirable to morcellate it into relatively smaller pieces of tissue, which are easier to remove. The present invention relates to an instrument and method for morcellating and removing a uterus.

US patent specifications U.S. Pat. Nos. 5,957,884, 6,007,512 and 6,036,681 describe morcellating devices in which an element carrying an electrode is rotated in order to cause the morcellation of tissue. This rotation of the electrode necessitates a mechanical drive arrangement, which increases the complexity of the instrument. The specification of our International patent application WO 2005/112806 seeks to provide a simpler and hence more reliable arrangement for the bulk removal of tissue, and provides the combination of a device for morcellating tissue within a body cavity of a patient and a tissue-pulling device. The morcellating device comprises a stationary tube having a distal end portion, the tissue-pulling device being locatable within the tube. The combination includes a bipolar electrosurgical electrode assembly including first and second electrodes located at the distal end of the tube and separated one from the other by an insulation member. The arrangement is such that, when an electrosurgical cutting voltage is applied to the electrode assembly, the tissue-pulling device can be moved to pull tissue against the distal end of the tube to form a core of severed tissue within the tube, and further moved in order to remove the severed tissue from the body cavity of the patient.

One problem with this known arrangement is that the morcellating device can be inserted too far into a patient's body, and this can lead to the morcellation of tissue which is not required to be morcellated. It can also lead to stalling of the morcellating device as it tries to morcellate too much tissue.

An aim of the invention is to provide a tissue morcellating device which can be accurately positioned within the body cavity of a patient to ensure morcellation of only tissue that needs to be morcellated.

### SUMMARY OF THE INVENTION

The present invention provides a tissue morcellating device for morcellating tissue within a body cavity of a patient, the morcellating device comprising a hollow tube having a distal end portion, and tissue cutting means located at the distal end of the tube, such that, when relative movement is initiated between the tube and the tissue, a core of severed tissue is formed within the tube for removal from the body cavity of the patient through the hollow tube, wherein the hollow tube is provided with a stop which is longitudinally adjustable with respect to the hollow tube, the stop being provided with a shoulder for limiting the longitudinal insertion of the tube into the patient's body.

Preferably, the stop is constructed in such a manner that, in a first configuration, it defines an internal aperture that is larger than the exterior of the tube, whereby the stop can freely move longitudinally with respect to the tube; and, in a second configuration, it defines an internal aperture which is sized for frictional engagement with the exterior of the tube, whereby the stop can be fixed to the tube in a predetermined position therealong.

In a preferred embodiment, the stop is constituted by an annular collar whose inner diameter is larger than the external diameter of the tube, the inner circumferencial surface of the collar being provided with a tapering cam track which houses a cam, the arrangement being such that, in a first position of the cam, it is positioned entirely within the cam track so that it does not engage with the external surface of the tube, thereby permitting the stop to move freely along the tube; and, in a second position of the cam, it extends inwardly with respect to the inner circumferential surface of the collar for frictional engagement with the external surface of the tube, thereby to fix the stop to the tube in a predetermined position therealong.

In another preferred embodiment, the stop is constituted by a cylindrical member and by a tubular member which is positioned around the cylindrical member for rotation relative thereto, a respective diametrical aperture being formed within each of the cylindrical member and the tubular member, the diameter of each of the diametrical apertures being greater than the diameter of the tube, wherein means are provided for biasing the tubular member circumferentially with respect to the cylindrical member into a position in which the diametrical apertures are out of alignment with one another, and wherein the stop is such that, in a first configuration, the diametrical apertures are aligned, whereby the stop is freely movable along the tube, and, in a second configuration, the diametrical apertures are out of alignment so as to define an effective central aperture which frictionally engages the external surface of the tube to fix the stop to the tube in a predetermined position therealong.

In yet another preferred embodiment, the stop is constituted by a V-shaped member made of resilient material, each arm of the V-shaped member being provided with an aperture whose diameter is greater than that of the tube, the free ends if the arms of the V-shaped member being manually engageable to move the free ends towards one another against the resilience of the V-shaped member, the arrangement being such that, in a first configuration in which no pressure is applied to the manually-engageable portions, the apertures in the V-shaped arms are sufficiently out of alignment for frictional engagement with the tube when the tube is positioned therethrough, thereby to fix the stop to the tube in a predetermined position therealong; and, in a second configuration, in which the manually-engageable portions are pressed together, the apertures are aligned to permit the stop to be moved freely along the tube.

In a further preferred embodiment, the stop is constituted by a length of resilient wire having manually-engageable end portions and a central portion constituted by two turns of a coil, the manually-engageable portions being movable towards one another to increase the size of the apertures so that, in a first position, in which the manual-engageable portions are pressed towards one another, the apertures are sufficiently opened up to permit the tube to slide freely relative thereto; and, in a second position in which the manually-engageable portions are not pressed towards one another, the apertures frictionally engage the tube to fix the stop to the tube in any desired position.

### BRIEF DESCRIPTION OF THE DRAWINGS

The invention will now be described in greater detail, by way of example, with reference to the drawings, in which:

FIG. 1 shows a morcellating device provided with a depth stop positioned in a non-operational (stored) position;

FIG. 2 shows the morcellating device with its depth stop in an operational position;

FIG. 3 is a perspective view of the operational part of the depth stop of FIGS. 1 and 2;

FIG. 4 is a partially broken-away perspective view of a second form of depth stop;

FIG. 5 is a perspective view of a third form of depth stop; and

FIG. 6 is a perspective view of a fourth form of depth stop.

### DESCRIPTION OF PREFERRED EMBODIMENTS

Referring to the drawings, FIG. 1 shows a morcellating device 1 comprising a handle 2 and a cylindrical tube 3. The cylindrical tube 3 is hollow, and defines a lumen 4 therein. The distal end of the tube 3 is provided with an electrosurgical electrode assembly 5. A tissue-pulling device (not shown) is insertable into the tube 3 from the proximal end 6 thereof, the distal end of the tissue-pulling device extending in use beyond the electrode assembly 5 at the distal end of the tube 3 for gripping tissue and pulling the tissue against the electrode assembly to form a core of severed tissue within the tube. The morcellating device 1 and the tissue-pulling device are described in greater detail in the specification of our International patent application WO 2005/112806.

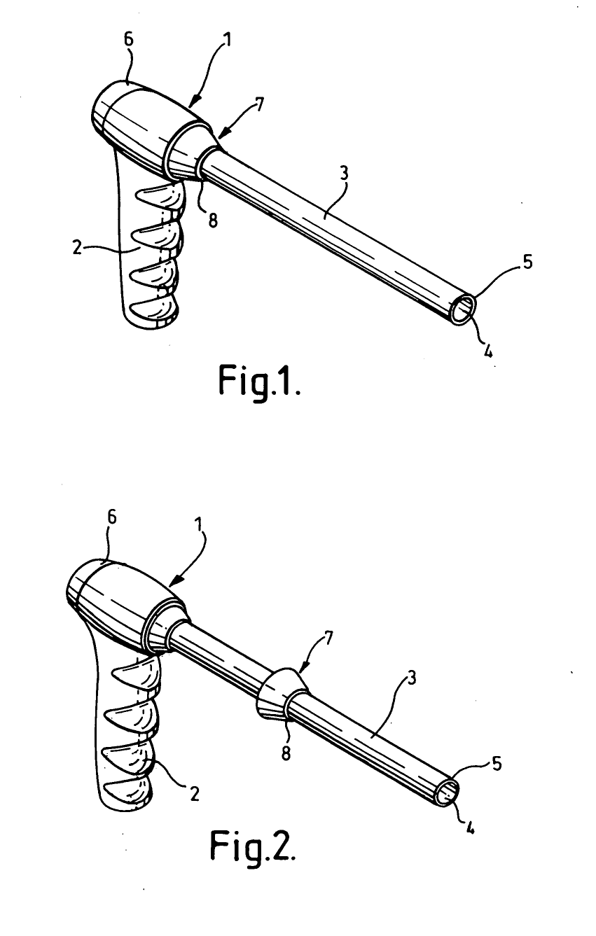

The morcellating device is provided with a depth stop 7 which can be fixed to the outer circumference of the tube 3 at any position along its length. FIG. 1 shows the depth stop 7 in its non-operational (stored) position, and FIG. 2 shows the depth stop in an operational position part way along the tube 3. The distal end of the depth stop 7 defines a shoulder 8 which limits the longitudinal insertion of the tube 3 into a patient's body.

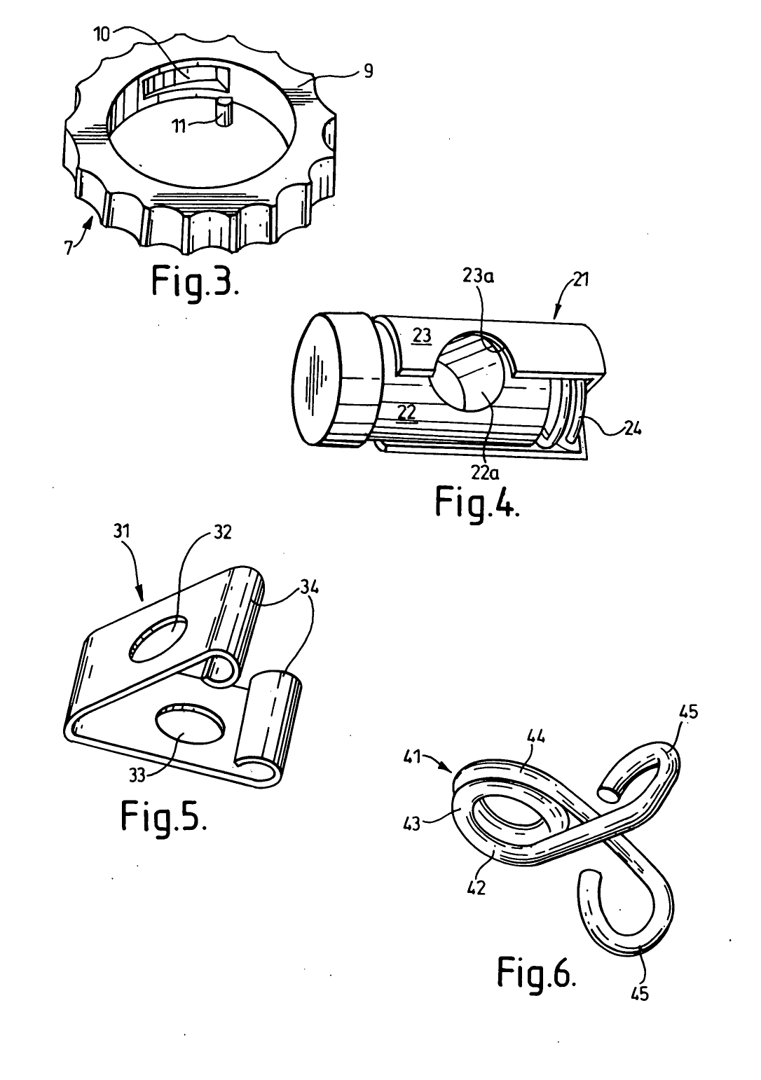

FIG. 3 shows the operational part of the depth stop 7, being constituted by a collar 9 having a cam track 10 provided on its inner circumference. A cam 11, in the form of the short cylindrical rod, is positioned within the cam track 10. Rotation of the depth stop 7 relative to the tube 3 causes the rod 11 to move from a first position in which the rod is located in the deepest part of the cam track 10, to a second position in which the rod is positioned at the shallow end of the cam track. In the first position, the rod 11 does not contact the outer circumference of the tube 3, and so the depth stop 7 is freely movable longitudinally with respect to the tube. In the second position, the rod 11 tightly engages the outer circumference of the tube 3, thereby locking the depth stop 7 to the tube.

It will be apparent that the depth stop 7 can be moved from the stored position shown in FIG. 1 to any required position along the tube 3, merely by rotating the depth stop to move the rod 11 into its first position, moving the depth stop to the required position along the tube, and rotating the depth stop in the opposite direction to force the rod into tight engagement with the outer circumference of the tube.

FIG. 4 shows a second form of depth stop 21, this depth stop being constituted by a generally cylindrical inner member 22 and a tubular outer member 23. The inner and outer members 22 and 23 are formed with respective diametrical apertures 22a and 23a. The diameter of each of the apertures of 22a and 23a is slightly larger than the outer diameter of the cylindrical tube 3 of the morcellating device 1. A spring 24 is provided to bias the outer member 23 circumferentially with respect to the inner member 22. Thus, when the depth stop 21 is positioned over the cylindrical tube 3, the spring 24 biases the outer member 23 so as to move the aperture 23a out of alignment with the aperture 22a, thereby defining an aperture passing through the depth stop which is of a smaller diameter than the external diameter of the tube 3. In this position, therefore, the depth stop 21 will firmly grip the tube 3, thereby locking the depth stop to the tube.

In order to position the depth stop 21 at any given position along the tube 3, the outer member 23 is rotated relative to the inner member 22, to bring the apertures 22a and 23a into alignment, thereby permitting the depth stop to be moved freely longitudinally along the tube until it reaches a desired depth stop position. The outer member 23 is then released, so that the spring 24 rotates the outer member 23 to cause the apertures 22a and 23 to be misaligned, thereby locking the depth stop 21 to the tube. In this embodiment, the outer circumferential surface of the outer member 23 defines a shoulder for limiting the longitudinal insertion of the tube 3 into a patient's body.

FIG. 5 shows a further form of depth stop 31, this depth stop being formed in a V-shape and made of spring metal material. Each arm of the V-shaped depth stop 31 is formed with a respective aperture 32, 33, each of which has a diameter which is larger than that of the tube 3. The free ends of the arms of the V-shaped depth stop 31 are formed with rolled-over portions 34 to constitute finger-engageable members.

In use, the finger-engageable members 34 of the depth stop 31 are firmly gripped to move them towards one another. In this position, the apertures 32 and 33 can be slid over the end of the tube 3, and the depth stop can be moved freely along the tube. In order to lock the depth stop 31 at any desired position along the tube 3, it is necessary only to release the pressure on the finger-engageable members 34, which causes the apertures 32 and 33 to move out of alignment and into firm engagement with the tube. In this embodiment, the distal V-shaped arm of the depth stop 31 defines a shoulder for limiting the longitudinal insertion of the tube 3 into a patient's body.

FIG. 6 shows a fourth form of depth stop 41. This depth stop 41 is made of a spring metal wire having a central portion 42, constituted by two turns of the spring wire. The two turns define apertures 43 and 44. The end portions of the spring wire are bent over to form finger-engageable members 45.

In the position shown, the apertures 43 and 44 in the two central turns of the spring wire are define a central aperture which has a diameter less than that of the tube 3. Consequently, the depth stop 41 can be fixed to the tube 3. In order to allow the depth stop 41 to be positioned on the tube 3, it is necessary to press the two finger-engageable members 45 towards one another, so as to open up the apertures 43 and 44. The depth stop 41 can then be positioned on the tube 3 and can be moved freely therealong. By releasing the pressure on the finger-engageable members 45, the depth stop 41 can then be locked to the tube 3 in any desired position. In this embodiment, the finger-engageable members 45 define a shoulder for limiting the longitudinal insertion of the tube 3 into a patient's body.

It will be apparent that modifications could be made to the morcellating device described above. In particular, other forms of depth stop could be utilised in place of the particular embodiments described. Thus, any suitable form of stop could be used, provided it is constructed in such a manner that, in a first configuration, it defines an internal aperture that is larger than the exterior of the tube, whereby the stop can freely move longitudinally with respect to the tube; and, in a second configuration, it defines an internal aperture which is sized for frictional engagement with the exterior of the tube, whereby the stop can be fixed to the tube in any predetermined position therealong. In particular, the depth stop could be a simple friction-fit ring.

It will also be appreciated that the any one of the depth stops described above could be used with any other form of endoscopic surgical instrument, so that the invention is not restricted to the use of a depth stop for a morcellator.

1. A tissue morcellating device for morcellating tissue within a body cavity of a patient, the morcellating device comprising a hollow tube having a distal end portion, and tissue cutting means located at the distal end of the tube, such that, when relative movement is initiated between the tube and the tissue, a core of severed tissue is formed within the tube for removal from the body cavity of the patient through the hollow tube, wherein the hollow tube is provided with a stop which is longitudinally adjustable with respect to the hollow tube, the stop being provided with a shoulder for limiting the longitudinal insertion of the tube into the patient's body.

2. A morcellating device as claimed in claim 1, wherein the stop is constructed in such a manner that, in a first configuration, it defines an internal aperture that is larger than the exterior of the tube, whereby the stop can freely move longitudinally with respect to the tube; and, in a second configuration, it defines an internal aperture which is sized for frictional engagement with the exterior of the tube, whereby the stop mechanism can be fixed to the tube in a predetermined position therealong.

3. A morcellating device as claimed in claim 2, wherein the stop is constituted by an annular collar whose inner diameter is larger than the external diameter of the tube, the inner circumferencial surface of the collar being provided with a tapering cam track which houses a cam, the arrangement being such that, in a first position of the cam, it is positioned entirely within the cam track so that it does not engage with the external surface of the tube, thereby permitting the stop to move freely along the tube; and, in a second position of the cam, it extends inwardly with respect to the inner circumferential surface of the collar for frictional engagement with the external surface of the tube, thereby to fix the stop to the tube in a predetermined position therealong.

4. A morcellating device as claimed in claim 2, wherein the stop is constituted by a cylindrical member and by a tubular member which is positioned around the cylindrical member for rotation relative thereto, a respective diametrical aperture being formed within each of the cylindrical member and the tubular member, the diameter of each of the diametrical apertures being greater than the diameter of the tube, wherein means are provided for biasing the tubular member circumferentially with respect to the cylindrical member into a position in which the diametrical apertures are out of alignment with one another, and wherein the stop is such that, in a first configuration, the diametrical apertures are aligned, whereby the stop is freely movable along the tube, and, in a second configuration, the diametrical apertures are out of alignment so as to define an effective central aperture which frictionally engages the external surface of the tube to fix the stop to the tube in a predetermined position therealong.

5. A morcellating device as claimed in claim 2, wherein the stop is constituted by a V-shaped member made of resilient material, each arm of the V-shaped member being provided with an aperture whose diameter is greater than that of the tube, the free ends if the arms of the V-shaped member being manually engageable to move the free ends towards one another against the resilience of the V-shaped member, the arrangement being such that, in a first configuration in which no pressure is applied to the manually-engageable portions, the apertures in the V-shaped arms are sufficiently out of alignment for frictional engagement with the tube when the tube is positioned therethrough, thereby to fix the stop to the tube in a predetermined position therealong; and, in a second configuration, in which the manually-engageable portions are pressed together, the apertures are aligned to permit the stop to be moved freely along the tube.

6. A morcellating device as claimed in claim 2, wherein the stop is constituted by a length of resilient wire having manually-engageable end portions and a central portion constituted by two turns of a coil, the manually-engageable portions being movable towards one another to increase the size of the apertures so that, in a first position, in which the manual-engageable portions are pressed towards one another, the apertures are sufficiently opened up to permit the tube to slide freely relative thereto; and, in a second position in which the manually-engageable portions are not pressed towards one another, the apertures frictionally engage the tube to fix the stop to the tube in any desired position.
